(19) United States
(12) Patent Application Publication
(10) Pub. No.: US 2010/0317868 A1
(43) Pub. Date: Dec. 16, 2010

(54) METHOD OF PREPARING TAXANE DERIVATIVES AND INTERMEDIATES USED THEREIN

(75) Inventors: Nam Du Kim, Hwaseong-si (KR); Wooseob Shin, Suwon-si (KR); Jaehyuk Jung, Goyang-si (KR); Dong Jun Kim, Osan-si (KR); Gi Jeong Kim, Suwon-si (KR); Young Ho Moon, Suwon-si (KR); Young-Kil Chang, Seoul (KR); Gwan Sun Lee, Seoul (KR); Tae Jin Choi, Goyang-si (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO., LTD., Kyungki-do (KR)

(21) Appl. No.: 12/517,980

(22) PCT Filed: Nov. 20, 2007
(86) PCT No.: PCT/KR2007/005829

§ 371 Date: Jun. 5, 2009

(30) Foreign Application Priority Data
Dec. 18, 2006 (KR) 10-2006-0129524

(51) Int. Cl. C07D 263/06; C07D 305/14

(52) U.S. Cl. 548/215; 549/510

(57) ABSTRACT

The present invention relates to a novel method of preparing a taxane derivative having an anti-tumor and anti-leukemia activity, and intermediates used therein.

### FIELD OF THE INVENTION

The present invention relates to a novel method of preparing a taxane derivative, and intermediates used therein.

### BACKGROUND OF THE INVENTION

Terpene taxane derivatives of formula (I) are potent anti-tumor chemotherapeutic agents having a broad spectrum of anti-tumor and anti-leukemia activity, some of which have been approved as commercially marketable therapeutic agents against ovarian cancer and breast cancer.

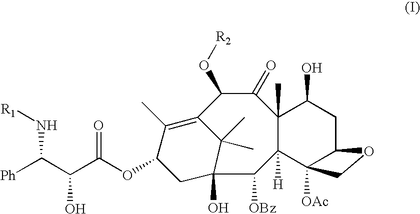

wherein,

Ph is phenyl;

Ac is acetyl;

Bz is benzoyl;

R₁ is t-butoxycarbonyl or benzoyl; and

R₂ is hydrogen or acetyl.

The preparation of the taxane derivatives such as docetaxel (R₁=t-butoxycarbonyl radical, R₂=H) and paclitaxel (R₁=benzoyl radical, R₂=Ac) involves the steps of selectively or simultaneously introducing protecting groups to hydroxy groups at 7- and 10-positions of 10-deacetylbaccatin III of formula (VIII) and protecting the hydroxy and amine groups of (2R,3S)-4-phenylisoserine.

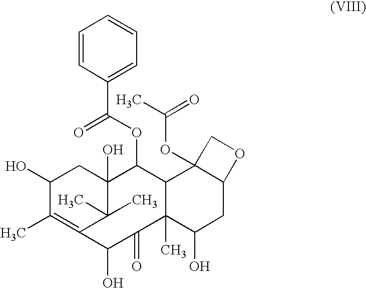

Desirable protecting groups of the hydroxyl groups at the 7- and 10-positions of 10-deacetylbaccatin III have been proposed to be various acyl groups such as 2,2,2-trichloroethoxycarbonyl, trichloroacetyl, dichloroacetyl, monochloroacetyl, t-butoxycarbonyl, 3,5-dinitrobenzoyl, and silyl groups such as triethylsilyl radical. Further, (2R,3S)-4-phenylisoserine requires protecting groups which can simultaneously protect the hydroxyl and amine groups thereof and can be easily removed after its coupling reaction with 10-deacetylbaccatin III having protected 7,10-hydroxyl groups. In this regard, oxazolidine and 13-lactam derivatives have been studied as a potentially viable protected (2R,3S)-4-phenylisoserine derivative.

For example, International Patent Publication WO 93/06094 discloses a process for preparing docetaxel by using a β-lactam derivative, but the synthesis of the β-lactam derivative itself is very difficult, and the coupling reaction must be conducted at a low temperature of −45° C. under an anhydrous condition.

Meanwhile, oxazolidine derivatives have also been widely studied. For example, Korean Patent Publication 93-702324 (International Patent Publication WO 91/09589) and International Patent Publication WO 02/12216 disclose an oxazolidine derivative of formula (IVa). However, during the preparation of the taxane derivative, the t-butoxycarbonyl radical is removed from the oxazolidine derivative of formula (IVa) when the ring opening reaction is carried out using formic acid after the coupling reaction, and accordingly, a t-butoxycarbonyl group must be reintroduced for the preparation of docetaxel, while a benzoyl group must be introduced in the case of paclitaxel preparation. Moreover, if an organic acid such as formic acid is present, the introduction of the t-butoxycarbonyl or benzoyl group may accompany significant side reactions.

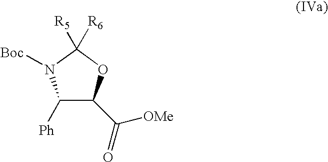

wherein,

Ph is phenyl;

Boc is t-butoxycarbonyl; and

R₅ and R₆ are each independently C₁₋₄ alkyl, C₁₋₄ alkyl substituted with one or more aryl groups, or aryl, while R₅ and R₆ may be optionally fused together with the carbon atom to which they are attached to form a 4- to 7-membered ring.

The synthetic procedures of oxazolidine derivatives of formula (IVb) and (IVc) disclosed in Korean Patent Publication 95-703546 (International Patent Publication WO 1994/07877) and Korean Patent Publication 95-703548 (International Patent Publication WO 1994/07879), respectively, are very complicated and they give low yields.

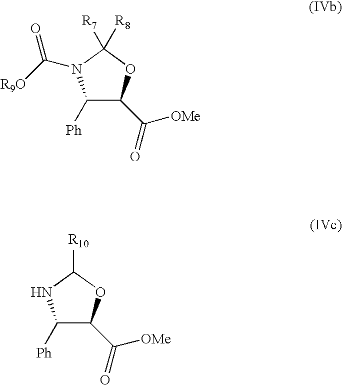

wherein,

Ph is phenyl;

R₇ and R₈ are each independently hydrogen, C₁₋₄ alkyl, C₂₋₄ alkenyl, or phenyl optionally substituted with one or more C₁₋₄ alkoxy radical, while R₇ and R₈ may be optionally fused together with the carbon atom to which they are attached to form a 4- to 7-membered ring;

R₉ is C₁₋₄ alkyl substituted with one or more Cl; and

R₁₀ is phenyl optionally substituted with trihalomethyl.

Further, a method for preparing the oxazolidine derivative of formula (IVd) is disclosed in Korean Patent Publication 95-703547 (International Patent Publication WO 1994/07878) as described in Reaction Scheme (I).

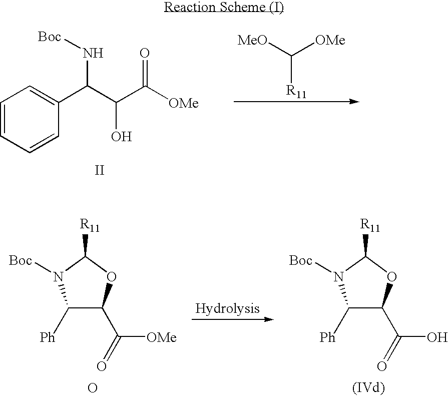

wherein,

Ph is phenyl; Boc is t-butoxycarbonyl; and

R₁₁ is hydrogen, or phenyl optionally substituted with one or more C₁₋₄ alkoxy.

When R₁₁ of formula (IVd) is a phenyl group substituted with an electron-donating substituent, e.g., p-methoxyphenyl radical, the demethylation reaction can be easily carried out under a mild condition without lossing the t-butoxycarbonyl group, as compared with other oxazolidine derivatives. However, the step of preparing the compound O from the compound of formula II in Reaction Scheme (II) is reversible, and this step gives a low yield of less than 70% when R₁₁ is a phenyl having an electron-donating substituent. This problem may arise from the fact that the access of proton or a nucleophile to the oxygen or nitrogen atom of the oxazolidine ring is easy due to the insufficient steric hindrance therearound.

In order to overcome the problems described above, the present inventors have attempted to prepare an oxazolidine derivative in a high yield by introducing thereto a naphthyl substituent, which exerts large steric hindrance to inhibit the access of proton or a nucleophile to the oxygen or nitrogen atom of the oxazolidine ring. Such a naphthalene substituent is capable of delocalizing more π-electrons than a phenyl group into the oxazolidine derivative, and have found a novel method for preparing a taxane derivative such as docetaxel and paclitaxel in high yields.

### SUMMARY OF THE INVENTION

Accordingly, it is an object of the present invention to provide a novel method preparing a taxane derivative such as docetaxel and paclitaxel, and intermediates used therein.

In accordance with one aspect of the present invention, there is provided a method for preparing a taxane derivative of formula (I), which comprises the steps of:

(i-a) reacting the compound of formula (II) with 1-dimethoxymethyl naphthalene in an organic solvent in the presence of an acid catalyst to obtain the oxazolidine methyl ester derivative of formula (III), and then (i-b) subjecting the obtained compound of formula (III) to hydrolysis under a basic condition to obtain the oxazolidic acid derivative of formula (IV) or a salt thereof;

(ii) subjecting the compound of formula (IV) or the salt thereof to a coupling reaction with a protected 10-deacetylbaccatin III of formula (V) in the presence of a condensation agent to obtain a taxane of formula (VI) having an oxazolidine side chain;

(iii) subjecting the compound of formula (VI) to a ring opening reaction in an organic solvent in the presence of an acid, followed by an optional step of replacing the t-butoxycarbonyl group of the resulting compound with a benzoyl group, to obtain a compound of formula (VII); and

(iv) removing at least one of protecting groups on the positions 7 and 10 of the compound of formula (VII):

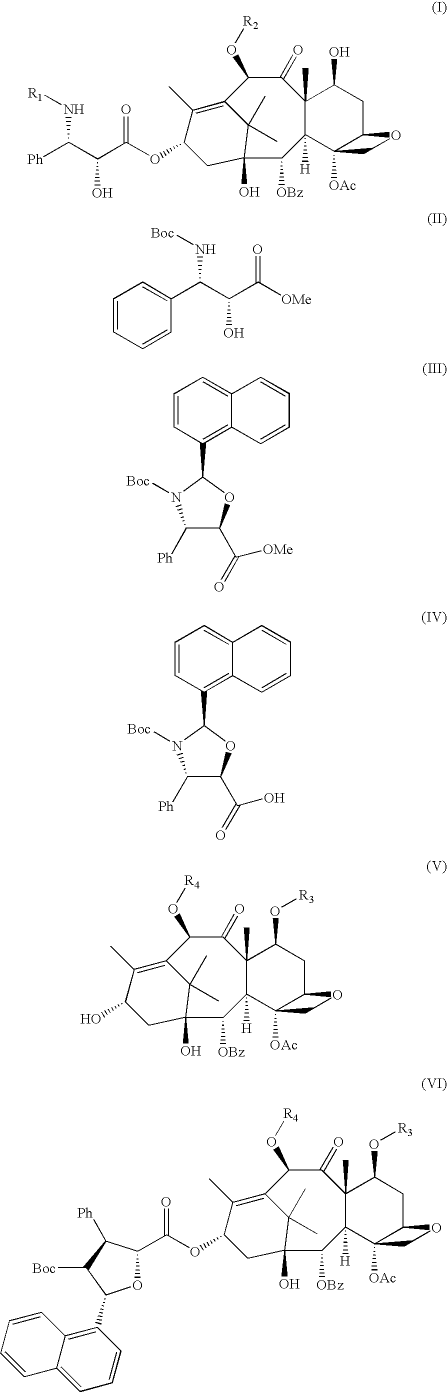

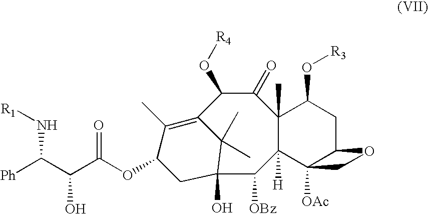

wherein,

Ph is phenyl;

Ac is acetyl;

Bz is benzoyl;

Boc is t-butoxycarbonyl;

R₁ is t-butoxycarbonyl or benzoyl;

R₂ is acetyl or hydrogen;

R₃ is a hydroxy protecting group which is 3,5-dinitrobenzoyl, trichloroacetyl, dichloroacetyl or 2,2,2-trichloroethoxycarbonyl; and

R₄ is R₃ or acetyl.

In accordance with another aspect of the present invention, there is provided the compound of formula (IV) used as an intermediate in preparing the taxane derivative of formula (I):

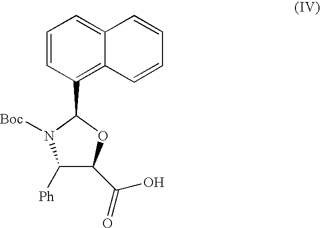

wherein,

Ph and Boc have same meanings as defined previously.

In accordance with further another aspect of the present invention, there is provided the compound of formula (VI) having the skeleton of the compound of formula (IV) incorporated as a side chain:

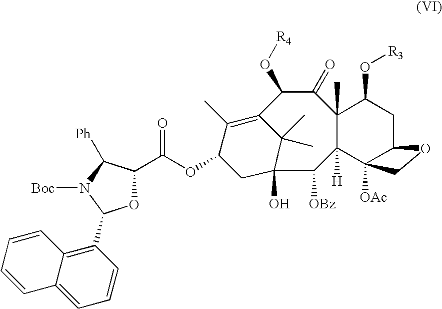

wherein,

Ph, Ac, Bz, Boc, R₃ and R₄ have same meanings as defined previously.

### DETAILED DESCRIPTION OF THE INVENTION

The method of preparing a taxane derivative according to the present invention is characterized by the use of both the oxazolidine derivative (formula (IV)) having a bulky naphthyl substituent which can create large steric hindrance therearound and a taxane compound (formula (VI)) having the oxazolidine derivative as an intermediate.

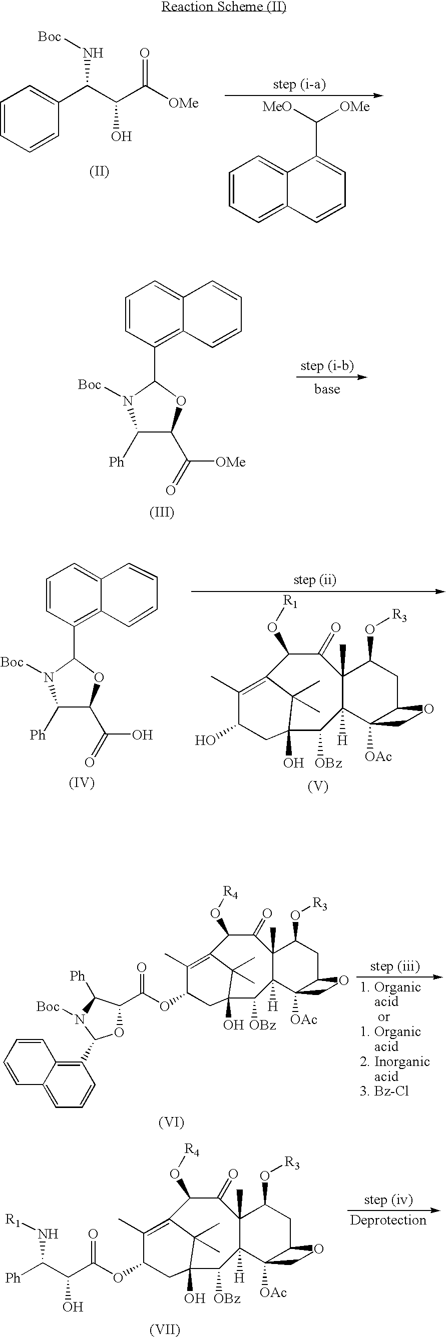

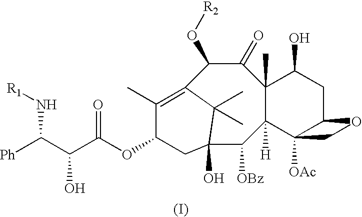

wherein,

Ph, Ac, Bz, Boc, R₁, R₂, R₃ and R₄ have the same meanings as defined previously.

The taxane derivative of formula (I) of the present invention, especially docetaxel or paclitaxel may be prepared by the procedure shown in Reaction Scheme (II), which is explained below in more detail.

In step (i-a), (2R,3S)—N-t-butoxycarbonyl-4-phenylisoserine methyl ester of formula (II) is allowed to react with 1-dimethoxymethylnaphthalene in an organic solvent in the presence of an acidic catalyst to obtain an oxazolidine methyl ester derivative of formula (III), which is then subjected to hydrolysis under a basic condition (step (i-b)), to obtain a novel oxazolidic acid derivative of formula (IV) in a high yield.

In this reaction, 1-dimethoxymethylnaphthalene may be used in an amount of 1 to 3 equivalents, preferably 1 to 1.5 equivalents based on (2R,3S)—N-t-butoxycarbonyl-4-phenylisoserine methyl ester (formula (II)). This reaction may be carried out at a temperature ranging from 0° C. to the boiling point of the solvent. The solvent used in this reaction may be toluene, hexane, cyclohexane, benzene, xylene or a mixture thereof, and the acid catalyst used in this reaction may be pyridinium p-toluenesulfonate, pyridinium 3-nitrobenzenesulfonate, pyridinium benzenesulfonate or a mixture thereof. The base used in hydrolysis may be an inorganic base such as lithium hydroxide, sodium hydroxide and potassium hydroxide, preferably lithium hydroxide. The inventive compound of formula (IV) may be used in the form of an amine-addition salt, and the amine is preferably triethylamine or pyridine.

In step (ii), the compound of formula (IV) obtained in step (i) or the salt thereof is subjected to a coupling reaction with a protected 10-deacetylbaccatin III of formula (V) in a solvent in the presence of a condensation agent to obtain a taxane derivative of formula (VI) having an oxazolidine side chain. This reaction may be carried out at a temperature ranging from 0° C. to 60° C., and the oxazolidic acid derivative of formula (IV) may be used in an amount of 1 to 5 equivalents based on the protected deacetylbaccatin III of formula (V). The solvent used in this reaction may be ethyl acetate, methyl acetate, chloroform, dichloromethane or tetrahydrofuran, and the condensation agent, e.g., dicyclohexylcarbodiimide, used in this reaction may be in an amount of 1 to 5 equivalents based on 10-deacetylbaccatin III.

Further, an activating agent such as 4-dimethylaminopyridine and pyridine may be added to the reaction mixture in a less than stoichiometric amount based on 10-deacetylbaccatin III.

Further, R₃, a protecting group of 10-deacetylbaccatin III, may be 3,5-dinitrobenzoyl, trichloroacetyl, dichloroacetyl or 2,2,2-trichloroethoxycarbonyl, and R₄ is identical with R₃ or acetyl.

In step (iii), the taxane derivative (formula (VI)) having an oxazolidine side chain obtained from step (ii) is subjected to a ring opening reaction to obtain a taxane derivative (formula (VII)) having protected 7- and 10-hydroxy groups, and the t-butoxycarbonyl group thereof is substituted with a benzoyl group. The acid used in the ring opening reaction may be hydrochloric acid, sulfuric acid, formic acid, p-toluenesulfonic acid, p-toluenesulfonic acid monohydrate and a mixture thereof, preferably p-toluenesulfonic acid monohydrate, in an amount of 0.1 to 30 equivalents based on the compound of formula (VI). The organic solvent used in this reaction may be chloroform, ethyl acetate, methyl acetate, dichloromethane, tetrahydrofuran, and a mixture thereof.

The ring opening reaction of oxazolidine carried out using only an acid catalyst and water does not proceed smoothly because of the hydrophobic nature around the oxazolidine ring. Therefore, an alcohol additive is used in place of a part of the water component to facilitate the ring opening reaction without generating undesirable side reactions. The alcohol that can be used for this purpose is C₁₋₃ alcohol, preferably methanol.

In order to synthesize paclitaxel, an additional step of replacing the t-butoxycarbonyl group with a benzoyl group is desirable. In this step, the t-butoxycarbonyl group is removed in the presence of hydrochloric acid, neutralized using a base such as sodium bicarbonate, and benzoyl chloride is added thereto, to obtain the compound of formula (VII), wherein R₄ is acetyl.

In step (iv), at least one of protecting groups at the positions 7 and 10 of the compound of formula (VII) is selectively removed to obtain the inventive taxane derivative. In this reaction, the protecting group can be removed by using an acid or base selected in accordance with the characteristics of the protecting group to be removed. For example, if R₃ or R₄ is 3,5-dinitrobenzoyl, trichloroacetyl or dichloroacetyl, a base such as morpholine, ammonia and ammonium acetate can be used in an amount of 1 to 40 equivalents based on the compound of formula (VII) to obtain the inventive taxane derivative. The solvent used in this reaction may be an alcohol, preferably C₁₋₃ alcohol, more preferably methanol. For example, if R₃ or R₄ is 2,2,2-trichloroethoxycarbonyl, an acid can be used to remove the protecting group in the presence of a zinc catalyst in accordance with the Korean Patent Publication 88-0001625 (European Patent Publication No. 0,253,738), to obtain the inventive taxane derivative.

In accordance with the method of the present invention, a taxane derivative, e.g., docetaxel or paclitaxel, can be prepared in a high yield and purity.

The following Examples are intended to further illustrate the present invention without limiting its scope.

### Example 1

### Preparation of (2R,4S,5R)-2-(1′-naphthyl)-3-t-butoxycarbonyl-4-phenyl-1,3-oxazolidine-5-carboxylic acid

### (1-1) Preparation of (2R,4S,5R)-2-(1′-naphthyl)-3-t-butoxycarbonyl-4-phenyl-1,3-oxazolidine-5-carboxylic acid methyl ester

29.5 g of (2R,3S)—N-t-butoxycarbonyl-4-phenylisoserine methyl ester, 0.6 g of pyridinium p-toluenesulfonate and 22.2 g of 1-dimethoxymethyl naphthalene were added dropwise to 600 ml of toluene, and the resulting solution was refluxed for 1 hour while removing 300 ml of toluene. The resulting solution was cooled to room temperature, diluted with 300 ml of ethyl acetate, and neutralized with 150 ml of saturated sodium bicarbonate. The organic layer was separated, washed with 150 ml of saturated salt solution and dried over anhydrous magnesium sulfate. The magnesium sulfate was filtered off and the organic solvent was removed under a reduced pressure to obtain the title compound (52 g).

¹H NMR (300 MHz, CDCl₃) d 8.36 (d, J=8.4 Hz, 1H), 7.89 (m, 2H, Ar), 7.42 (m, 10H, Ar), 5.60 (s, 1H), 4.58 (d, J=2.7 Hz, 1H), 3.11 (s, 3H), 1.06 (s, 9H).

### (1-2) Preparation of (2R,4S,5R)-2-(1′-naphthyl)-3-t-butoxycarbonyl-4-phenyl-1,3-oxazolidine-5-carboxylic acid

A solution obtained by dissolving the compound obtained in (1-1) in 500 ml of methanol was stirred at 0° C. for 2 hours while slowly adding 60 ml of 3N lithium hydroxide dropwise thereto. 25 ml of methanol was removed from the resulting mixture under a reduced pressure, and 25 ml of water was added dropwise thereto. The water layer of the resulting mixture was washed twice with 100 ml portions of ethyl acetate/hexane (1/10 (v/v)), and the resulting mixture was neutralized by slowly adding dropwise thereto 20 ml of 3N hydrochloric acid while keeping the temperature of the mixture at 0° C. Then, 100 ml of ethyl acetate was added dropwise thereto. After removing the water layer, the organic layer was washed with 100 ml of saturated NaCl and dried over anhydrous magnesium sulfate. The magnesium sulfate was filtered off and the organic solvent was removed under a reduced pressure to obtain the title compound (41.3 g, 98.5%).

b.p.: 119° C.;

[a]_{D}²³=+56.9° (c=1, CHCl₃); and

¹H NMR (300 MHz, CDCl₃) d 8.32 (d, J=8.3 Hz, 1H), 7.91 (m, 2H, Ar), 7.46 (m, 10H, Ar), 5.60 (d, J=3.1 Hz, 1H), 4.62 (d, J=3.1 Hz, 1H), 1.04 (s, 9H).

### Example 2

### Preparation of 13-[(2′R,4′S,5′R)-3′-t-butoxycarbonyl-2′-(1′″-naphthyl)-4-phenyl-1′,3′-oxazolidine-5′-carbonyl]-7,10-(di-3″,5″-dinitrobenzoyl)-10-deacetylbaccatin III

A solution obtained by dissolving 9.2 g of (2R,4S,5R)-2-(1′-naphthyl)-3-t-butoxycarbonyl-4-phenyl-1,3-oxazolidine-5-carboxylic acid obtained in Example 1, 9.3 g of 7,10-(di-3′,5′-dinitrobenzoyl)-10-deacetylbaccatin III and 61 ml of 4-(dimethylamino)pyridine in 180 ml of ethyl acetate was stirred while keeping the temperature of the solution 30° C. 5.2 g of dicyclohexylcarbodiimide was added thereto at 40° C. and stirred for 30 min, followed by filtering the resulting dicyclohexylurea. The cake was washed with 20 ml of ethyl acetate, and the combined organic layer was washed sequentially with 30 ml of 1N hydrochloric acid and 30 ml of saturated sodium bicarbonate, and dried over anhydrous magnesium sulfate. The magnesium sulfate was filtered off and the organic solvent was removed under a reduced pressure. 80 ml of acetonitrile was added to the resulting residue and stirred for 1 hour, and 80 ml of water was slowly added dropwise thereto and stirred for 2 hours. The solid was filtered, and the procedure of adding and stirring each of 80 ml of acetonitrile and water was repeated as described above. The resulting solid was filtered to obtain the title compound (13.4 g, 100%).

b.p.: 202° C.;

[a]_{D}²³=−16.1° (c=1, CHCl₃);

IR (KBr, cm⁻¹) 3560, 3446, 3102, 2977, 2939, 2897, 1740, 1718, 1628, 1548, 1547, 1344, 1268, 1162, 1069, 978, 919, 729, 718; and

¹H NMR (300 MHz, CDCl₃) d 9.27 (m, 1H), 9.20 (m, 1H), 9.04 (m, 2H), 8.76 (m, 2H), 8.11 (d, J=7.5 Hz, 2H), 8.02 (m, 2H), 7.62 (m, 2H), 7.53-7.43 (m, 13H), 6.30 (s, 1H), 5.95 (t, J=8.3 Hz, 1H), 5.68-5.58 (m, 3H), 4.93 (d, J=8.0 Hz), 4.68 (d, J=4.3 Hz), 4.32 (d, J=8.6 Hz, 1H), 4.14 (d, J=8.6 Hz, 1H), 3.79 (d, J=7.1 Hz, 1H), 2.83-2.79 (m, 1H), 2.20-1.98 (m, 6H), 1.90 (s, 3H), 1.56 (s, 3H), 1.25 (s, 3H), 1.19 (s, 3H), 0.86 (s, 12H).

### Example 3

### Preparation of 13-[(2′R,4′S,5′R)-3′-t-butoxycarbonyl-2′-(1′″-naphthyl)-4′-phenyl-1′,3′-oxazolidine-5′-carbonyl]-7,10-(di-2″,2″,2″-trichloroethoxycarbonyl)-10-deacetylbaccatin III

The procedure of Example 2 was repeated except for using 7,10-(di-2′,2′,2′-trichloroethoxycarbonyl)-10-deacetylbaccatin III as a taxane derivative of formula (V) to obtain the title compound (14.0 g).

¹H NMR (300 MHz, CDCl₃) d 8.36 (d, J=8.4 Hz, 1H), 8.02 (d, J=8.4 Hz, 2H), 7.86 (d, J=8.2 Hz, 2H), 7.30-7.62 (m, 13H), 5.95 (s, 1H), 5.92 (m, 1H), 5.60 (m, 1H), 5.62 (d, J=4.5 Hz, 1H), 5.58 (d, J=7.0 Hz, 1H), 5.35 (m, 1H), 4.87 (d, J=11.8 Hz, 1H), 4.83 (d, J=8.1 Hz, 1H), 4.76 (s, 2H), 4.64 (d, J=4.6 Hz, 1H), 4.58 (d, J=11.8 Hz, 1H), 4.23 (d, J=8.5 Hz), 4.06 (d, J=8.4 Hz, 1H), 3.66 (d, J=6.8 Hz, 1H), 2.55-2.70 (m, 1H), 2.20-2.25 (m, 1H), 2.00-2.10 (m, 1H), 1.88 (s, 3H), 1.75 (s, 3H), 1.61 (s, 1H), 1.55 (s, 3H), 1.10 (s, 3H), 1.03 (s, 3H), 0.96 (s, 9H).

### Example 4

### Preparation of 13-[(2′,3′S)-3′-t-butoxycarbonylamino-3′-phenyl-2′-hydroxypropinonyl]-10-deacetylbaccatin III

### (4-1) Preparation of 13-[(2′R,3′S)-3′-t-butoxycarbonylamino-3′-phenyl-2′-hydroxypropinonyl]-7,10-(di-3″,5″-dinitrobenzoyl)-10-deacetylbaccatin III

13.4 g of 13-[(2′R,4′S,5′R)-3′-t-butoxycarbonyl-2′-(1′″-naphthyl)-4′-phenyl-1′,3′-oxazolidine-5′-carbonyl]-7,10-(di-3″,5″-dinitrobenzoyl)-10-deacetylbaccatin III obtained in Example 2 was dissolved in a mixture of 67 ml of chloroform and 13 ml of methanol. 1.92 g of p-toluenesulfonic acid monohydrate was added dropwise thereto and stirred at room temperature for 3 hours. The organic layer was washed with 135 ml of water containing 1.3 g of sodium bicarbonate and dried over anhydrous magnesium sulfate. The magnesium sulfate was filtered off and the organic solvent was removed under a reduced pressure. The obtained solid was dissolved in 120 ml of diethyl ether, and 240 ml of hexane was slowly added dropwise thereto and stirred at room temperature for 3 hours. The mixture was filtered and the obtained solid was dissolved in 33 ml of acetonitrile, followed by slowly adding 77 ml of water dropwise thereto. The resulting solution was stirred at room temperature for 3 hours, and the solvent was removed under a reduced pressure to obtain the title compound (10.9 g, 91%).

b.p.: 173° C.;

[a]_{D}²³=−8.9° (c=1, CHCl₃);

IR (KBr, cm⁻¹) 3543, 3432, 3101, 2978, 2900, 1736, 1628, 1548, 1494, 1455, 1368, 1345, 1269, 1163, 1095, 1070, 978, 920, 730, 718; and

¹H-NMR (CDCl₃, 300 MHz): d 9.27 (m, 1H), 9.21 (m, 1H), 9.03 (m, 2H), 8.87 (m, 2H), 8.15 (d, J=7.5 Hz, 2H), 7.65 (m, 1H), 7.54 (m, 2H), 7.40-7.43 (m, 5H), 6.63 (s, 1H), 6.27 (m, 1H), 5.88 (m, 1H), 5.80 (d, J=6.9 Hz, 1H), 5.38 (d, J=9.4 Hz, 1H), 5.28 (m, 1H), 5.03 (d, J=8.1 Hz, 1H), 4.67 (d, J=3.1 Hz, 1H), 4.41 (d, J=8.6 Hz, 1H), 4.26 (d, J=8.6 Hz, 1H), 4.07 (d, J=6.7 Hz, 1H), 3.34 (d, J=5.3 Hz, 1H), 2.87 (m, 1H), 2.46 (s, 3H), 2.42 (m, 2H), 2.01-2.05 (m, 3H), 2.01 (s, 3H), 1.87 (s, 1H), 1.59 (s, 3H), 1.39 (s, 3H), 1.36 (s, 9H), 1.32 (s, 3H).

### (4-2) Preparation of 13-[(2′R,3′S)-3′-t-butoxycarbonylamino-3′-phenyl-2′-hydroxypropinonyl]-10-deacetylbaccatin III

6.0 g of 13-[(2′R,3′S)-3′-t-butoxycarbonylamino-3′-phenyl-2′-hydroxypropinonyl]-7,10-(di-3″,5″-dinitrobenzoyl)-10-deacetylbaccatin III obtained in (4-1) was added to a mixture of 30 ml of methanol and 6 ml of morpholine, and stirred at room temperature for 3 hours. 50 ml of ethyl acetate was added dropwise thereto, and then 70 ml of 1N hydrochloric acid was slowly added dropwise thereto at 0° C. The organic layer was separated and dried over anhydrous magnesium sulfate. The magnesium sulfate was filtered off and the organic solvent was removed under a reduced pressure. The resulting residue was subjected to silica column chromatography to obtain the title compound as a white solid (3.5 g, 87%).

b.p.: 195° C.;

[a]_{D}²³=−43.9° (c=0.74, ethanol);

IR (KBr, cm⁻¹) 3652, 3487, 3367, 2978, 2936, 2903, 1711, 1603, 1498, 1367, 1267, 1244, 1175, 1093, 1071, 1023, 976, 896, 709; and

¹H NMR (300 MHz, CDCl₃) d 8.11 (d, J=7.2 Hz, 2H), 7.61 (m, 1H), 7.51 (m, 2H), 7.28-7.42 (m, 5H), 6.23 (m, 1H), 5.69 (d, J=7.0 Hz, 1H), 5.45 (d, J=9.6 Hz, 1H), 5.29 (m, 1H), 5.22 (s, 1H), 4.96 (m, 1H), 4.64 (m, 1H), 4.33 (d, J=8.4 Hz, 1H), 4.19-4.24 (m, 3H), 3.93 (d, J=6.9 Hz, 1H), 3.37 (d, J=5.4 Hz, 1H), 2.56-2.65 (m, 1H), 2.39 (s, 3H), 2.27-3.1 (m, 2H), 1.82-1.91 (m, 1H), 1.86 (s, 3H), 1.78 (s, 3H), 1.70 (s, 1H), 1.54 (b, 1H), 1.36 (s, 9H), 1.26 (s, 3H), 1.15 (s, 9H).

### Example 5

### Preparation of 13-[(2′R,3′S)-3′-t-butoxycarbonylamino-3′-phenyl-2′-hydroxypropinonyl]-10-deacetylbaccatin III

(5-1) Preparation of 13-[(2′R,3′S)-3′-t-butoxycarbonylamino-3′-phenyl-2′-hydroxypropinonyl]-7,10-(di-2″,2″,2″-trichloroethoxycarbonyl)-10-deacetylbaccatin III

14 g of 13-[(2′R,4′S,5′R)-3′-t-butoxycarbonyl-2′-(1′″-naphthyl)-4′-phenyl-1′,3′-oxazolidine-5′-carbonyl]-7,10-(di-2″,2″,2″-trichloroethoxycarbonyl)-10-deacetylbaccatin III obtained in Example 3 was dissolved in 130 ml of chloroform. 1.92 g of p-toluenesulfonic acid monohydrate was added dropwise thereto and stirred at room temperature for 3 hours. The organic layer was washed with 130 ml of water containing 13 g of sodium bicarbonate and dried over anhydrous magnesium sulfate. The magnesium sulfate was filtered off and the organic solvent was removed under a reduced pressure. The obtained solid was subjected to column chromatography to obtain the title compound (10.2 g, 88%). Analytic and spectrometric spectroscopic data of the compound were identical with those of the material reported in European Patent Publication No. 0,253,738.

### (5-2) Preparation of 13-[(2′R,3′S)-3′-t-butoxycarbonylamino-3′-phenyl-2′-hydroxypropinonyl]-10-deacetylbaccatin III

The procedure of European Patent Publication No. 0,253,738 was repeated except for using the compound obtained in (5-1) as a starting material to obtain docetaxel as a title compound (6.4 g, 90%). Analytic and spectrometric data of the compound were identical with those of the compound of Example 4.

### Example 6

### Preparation of 7-trichloroacetylbaccatin III

10 g of 10-deacetylbaccatin III was dissolved in a mixture of 40 ml of pyridine and 300 ml of CHCl₃, and stirred for 10 min. 2.46 ml of trichloroacetyl chloride dissolved in 50 ml of CHCl₃ was added dropwise thereto at 35° C. for 3 hours and stirred for 1 hour. Then, 3.28 ml of acetyl bromide dissolved in 25 ml of CHCl₃ was slowly added dropwise thereto for 2 hours and stirred at room temperature for 3 hours. After completing the reaction, the reaction solution was neutralized by slowly adding 100 ml of water and 40 ml of concentrated HCl thereto, which was extracted with CHCl₃. The organic layer was treated with MgSO₄ and filtered. The organic solvent was removed from the filtered solution under a reduced pressure to obtain the title compound (13.4 g, 100%).

b.p.: 180° C.;

[a]_{D}²³=62.3° (c=0.74, ethanol); and

¹H NMR (CHCl₃) d: 8.11 (2H, d, J=7.3), 7.62 (1H, t, J=7.4), 7.49 (2H, t, J=7.8), 6.43 (1H, s), 5.74˜5.65 (2H, m), 5.00 (1H, d, J=7.9), 4.93˜4.80 (1H, m), 4.35 (1H, d, J=8.3), 4.17 (1H, d, J=8.2), 4.04 (1H, d, J=6.7), 2.80˜2.63 (1H, m), 2.35˜2.29 (5H, m), 2.16˜2.14 (7H, m), 2.01-1.97 (1H, m), 1.87 (3H, s), 1.59 (1H, s), 1.13 (3H, s), 1.09 (3H, s).

### Example 7

### Preparation of 13-[(2′R,4′S,5′R)-3′-t-butoxycarbonyl-2′-(1″-naphthyl)-4′-phenyl-1′,3′-oxazolidine-5′-carbonyl]-7-trichloroacetylbaccatin III

13.4 g of 7-trichloroacetylbaccatin III obtained in Example 6, 9.25 g of (2R,4S,5R)-2-(1′-naphthyl)-3-t-butoxycarbonyl-4-phenyl-1,3-oxazolidine-5-carboxylic acid obtained in (1-2) of Example 1 and 100 mg of 4-(dimethylamino)pyridine were dissolved in 134 ml of ethyl acetate. 5.64 g of dicyclohexylcarbodiimide was added thereto at room temperature and stirred for 1 hour, followed by filtering the resulting dicyclohexylurea. The cake was washed with 20 ml of ethyl acetate, and the combined organic layer was sequentially washed with 30 ml of 1N hydrochloric acid and 30 ml of saturated sodium bicarbonate, and dried over anhydrous magnesium sulfate. The magnesium sulfate was filtered off and the organic solvent was removed under a reduced pressure to obtain the title compound (20.8 g, 100%).

¹H NMR (CDCl₃) d: 8.35 (1H, d, J=8.1), 8.02 (2H, d, J=7.4), 7.90 (2H, t, J=7.8), 7.66˜7.44 (12H, m), 7.25 (1H, bs), 6.11 (1H, s), 5.92 (1H, bs), 5.66˜5.60 (2H, m), 5.43 (1H, t, J=6.5), 4.84 (1H, d, J=8.1), 4.66 (1H, d, J=4.5), 4.27 (1H, d, J=8.4), 4.09 (1H, d, J=8.3), 3.73 (1H, d, J=6.9), 2.72-2.54 (1H, m), 2.14 (3H, s), 2.07˜1.80 (7H, m), 1.62˜1.58 (6H, m), 1.14 (3H, s), 1.08 (3H, s), 0.99 (9H, s).

### Example 8

### Preparation of 13-[(2′R,3′S)-3′-t-butoxycarbonylamino-3′-phenyl-2′-hydroxypropinonyl]-7-trichloroacetylbaccatin III

20.8 g of 13-[(2′R,4′S,5′R)-3′-t-butoxycarbonyl-2′-(1′″-naphthyl)-4′-phenyl-1′,3′-oxazolidine-5′-carbonyl]-7-trichloroacetylbaccatin III obtained in Example 7 was dissolved in a mixture of 100 ml of CHCl₃ and 20 ml of MeOH, and 3.7 g of p-toluenesulfonic acid monohydrate was added dropwise thereto. The solution was stirred for 5 hours, neutralized by adding 100 ml of saturated sodium bicarbonate dropwise thereto, and the resulting solution was extracted twice with CHCl₃. The organic layer was dried over anhydrous magnesium sulfate. The magnesium sulfate was filtered off and the organic solvent was removed under a reduced pressure. The resulting residue was subjected to column chromatography to obtain the title compound (13.7 g, 75%).

¹H NMR (CDCl₃) d: 8.11 (2H, d, J=7.2), 7.62 (1H, t, J=7.4), 7.51 (2H, t, J=7.7), 7.41˜7.35 (5H, m), 6.41 (1H, s), 6.23˜6.15 (1H, t, J=7.0), 5.72˜5.65 (2H, m), 5.40 (1H, d, J=9.5), 5.27˜5.30 (1H, bd), 4.95 (1H, d, J=8.2), 4.64 (1H, bs), 4.34 (1H, d, J=8.6), 4.20 (1H, d, J=8.4), 3.97 (1H, d, J=6.6), 3.39 (1H, d, J=5.4), 2.74˜2.65 (1H, m), 2.40 (3H, s), 2.33 (2H, d, J=9.1), 2.17 (3H, s), 2.04-1.88 (7H, m), 1.75 (1H, s), 1.35 (9H, s), 1.23 (3H, s), 1.18 (3H, s).

### Example 9

### Preparation of 13-[(2′R,3′S)-3′-benzoylamino-3′-phenyl-2′-hydroxypropinonyl]-7-trichloroacetylbaccatin III

13.7 g of 13-[(2′R,3′S)-3′-t-butoxycarbonylamino-3′-phenyl-2′-hydroxypropinonyl]-7-trichloroacetylbaccatin III obtained in Example 8 was dissolved in 140 ml of MeOH. 35 ml of 3N HCl was added dropwise thereto and stirred at 50-55° C. for 4 hours. The reaction solution was cooled to room temperature. 30 ml of ethyl acetate and 30 ml of saturated sodium bicarbonate were added dropwise thereto, and 2.0 ml of benzoyl chloride was further added dropwise thereto. The resulting solution was stirred for 1 hour and extracted twice with 30 ml of ethyl acetate. The organic layer was washed with 50 ml of saturated salt and dried over anhydrous magnesium sulfate. The magnesium sulfate was filtered off and the organic solvent was removed therefrom under a reduced pressure. The resulting residue was subjected to column chromatography to obtain the title compound (11 g, 80%).

¹H NMR (CDCl₃) d: 8.11 (2H, d, J=7.1), 7.74 (2H, d, J=7.1), 7.61˜7.35 (11H, m),7.13 (1H, d, J=7.8), 6.37 (1H, s), 6.22-6.15 (1H, t, J=7.3), 5.87˜5.61 (3H, m), 4.98 (1H, d, J=7.9), 4.80 (1H, s), 4.32 (1H, d, J=8.3), 4.21 (1H, d, J=8.4), 3.95 (1H, d, J=6.7), 3.85 (1H, bs), 2.75˜2.65 (1H, m), 2.41 (3H, s), 2.35 (2H, d, J=9.0), 2.17 (3H, s), 1.99˜1.97 (2H, m), 1.88 (6H, d, J=7.0), 1.20 (3H, s), 1.15 (3H, s).

### Example 10

### Preparation of 13-[(2′R,3′S)-3′-benzoylamino-3′-phenyl-2′-hydroxypropinonyl]-baccatin III

11.0 g of 13-[(2′R,3′S)-3′-benzoylamino-3′-phenyl-2′-hydroxypropinonyl]-7-trichloroacetylbaccatin III obtained in Example 9 was dissolved in a mixture of 30 ml of THF and 30 ml of MeOH. 2.5 g of ammonium acetate was added thereto and stirred for 4 hours. The solvent was removed from the solution under a reduced pressure, 60 ml of water was added dropwise thereto, and the resulting solution was extracted twice with 60 ml of ethyl acetate. The organic layer was washed with 100 ml of saturated salt and dried over anhydrous magnesium sulfate. The magnesium sulfate was filtered off and the organic solvent was removed under a reduced pressure. The resulting residue was subjected to column chromatography to obtain the title compound (7.5 g, 80%).

b.p.: 218-222° C.;

[a]_{D}²³=54.6° (c=1.0, methanol);

IR (KBr, cm⁻¹) 3510.6, 3440.2, 2962.7, 2944.5, 1735.0, 1712.8, 1646.5, 1580.4, 1541.4, 1513.7, 1481.9, 1451.5, 1436.0, 1408.9, 1370.3, 1346.9, 1317.2, 1244.2, 1176.7, 1146.4, 1108.8, 1096.6, 1072.9, 1025.2, 985.0, 966.5, 945.3, 709.7; and

¹H NMR (CDCl₃) d: 8.13 (2H, d, J=7.1), 7.73 (2H, d, J=7.1), 7.63˜7.34 (11H, m), 7.00 (1H, d, J=8.7), 6.27 (1H, s), 6.23 (1H, t, J=9.4), 5.80 (1H, d, J=8.9), 5.67 (1H, d, J=7.1), 4.90 (1H, d, J=8.1), 4.78 (1H, d, J=5.3), 4.40-4.34 (1H, m), 4.30 (1H, d, J=8.2), 4.20 (1H, d, J=8.7), 3.79 (1H, d, J=6.8), 3.59 (1H, d, J=5.2), 2.65˜2.47 (2H, m), 2.38˜2.32 (5H, m), 2.23 (3H, s), 1.93˜1.85 (2H, m), 1.80 (3H, s), 1.60 (3H, s), 1.23 (3H, s), 1.14 (3H, s).

While the invention has been described with respect to the above specific embodiments, it should be recognized that various modifications and changes of the invention also fall within the scope of the present invention defined by the claims that follow.

What is claimed is:

1. A method for preparing a taxane derivative of formula (I), which comprises the steps of:
(i-a) reacting the compound of formula (II) with 1-dimethoxymethyl naphthalene in an organic solvent in the presence of an acid catalyst to obtain the oxazolidine methyl ester derivative of formula (III), and then (i-b) subjecting the obtained compound of formula (III) to hydrolysis under a basic condition to obtain the oxazolidic acid derivative of formula (IV) or a salt thereof;
(ii) subjecting the compound of formula (IV) or the salt thereof to a coupling reaction with a protected 10-deacetylbaccatin III of formula (V) in the presence of a condensation agent to obtain a taxane of formula (VI) having an oxazolidine side chain;
(iii) subjecting the compound of formula (VI) to a ring opening reaction in an organic solvent in the presence of an acid, followed by an optional step of replacing the t-butoxycarbonyl group of the resulting compound with a benzoyl group, to obtain a compound of formula (VII); and
(iv) removing at least one of protecting groups on the positions 7 and 10 of the compound of formula (VII): 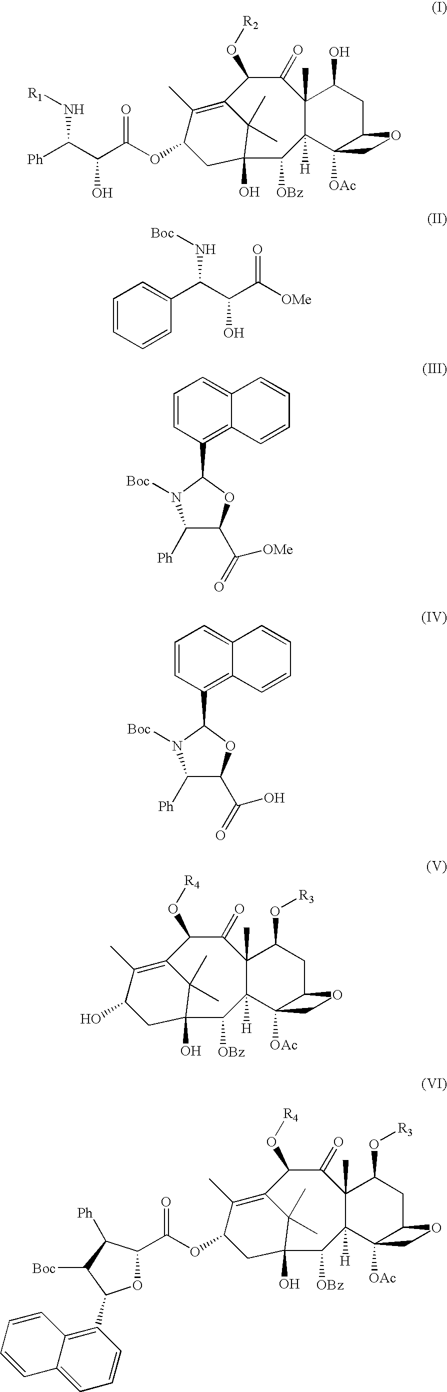 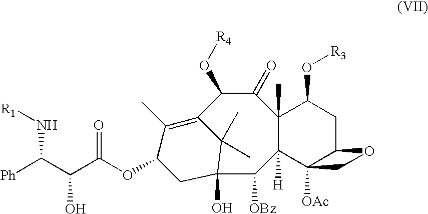
wherein,
Ph is phenyl;
Ac is acetyl;
Bz is benzoyl;
Boc is t-butoxycarbonyl;
R₁ is t-butoxycarbonyl or benzoyl;
R₂ is acetyl or hydrogen;
R₃ is a hydroxy protecting group which is 3,5-dinitrobenzoyl, trichloroacetyl, dichloroacetyl or 2,2,2-trichloroethoxycarbonyl; and
R₄ is R₃ or acetyl.

2. The method of claim 1, wherein 1-dimethoxymethylnaphthalene in step (i-a) is used in an amount ranging from 1 to 3 equivalents based on the compound of formula (II).

3. The method of claim 1, wherein the acid catalyst used in step (i-a) is selected from the group consisting of pyridinium p-toluenesulfonate, pyridinium 3-nitrobenzenesulfonate, pyridinium benzenesulfonate, and a mixture thereof.

4. The method of claim 1, wherein the base used in hydrolysis in step (i-b) is selected from the group consisting of lithium hydroxide, sodium hydroxide, potassium hydroxide, and a mixture thereof.

5. The method of claim 1, wherein the oxazolidic acid derivative of formula (IV) used in step (ii) is used in an amount ranging from 1 to 5 equivalents based on the protected deacetylbaccatin III of formula (V).

6. The method of claim 1, wherein the condensation agent used in step (ii) is dicyclohexylcarbodiimide.

7. The method of claim 1, wherein 4-dimethylaminopyridine or pyridine is further added during step (ii) as an activating agent.

8. The method of claim 1, wherein the acid used in step (iii) is selected from the group consisting of hydrochloric acid, sulfuric acid, formic acid, p-toluenesulfonic acid, p-toluenesulfonic acid monohydrate, and a mixture thereof.

9. The method of claim 1, wherein the acid in step (iii) is used in an amount ranging from 1 to 30 equivalents based on the compound of formula (VI).

10. The method of claim 1, wherein C₁₋₃ alcohol is further added to the ring opening of step (iii).

11. A compound of formula (IV): 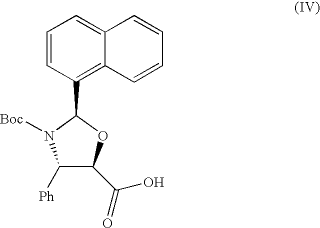
wherein,
Boc and Ph have same meanings as defined in claim 1.

12. A compound of formula (VI): 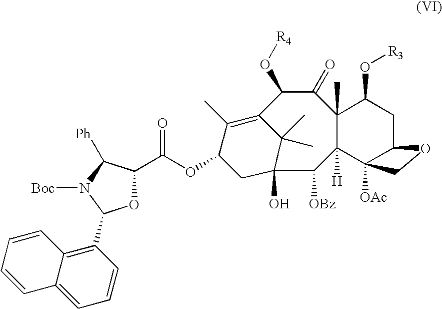
wherein,
Ph, Ac, Bz, Boc, R₃ and R₄ have same meanings as defined in claim 1.
